(19) United States
(12) Patent Application Publication
(10) Pub. No.: US 2019/0076496 A1
(43) Pub. Date: Mar. 14, 2019

(54) POLYSACCHARIDE DIGESTION INHIBITOR

(71) Applicant: Q' SAI CO., LTD., Fukuoka (JP)

(72) Inventors: Yosuke INAGAKI, Fukuoka (JP); Mihoko KUROKAWA, Fukuoka (JP)

(21) Appl. No.: 16/084,509

(22) PCT Filed: Mar. 14, 2017
(86) PCT No.: PCT/JP2017/010193

§ 371 Date: Sep. 12, 2018

(30) Foreign Application Priority Data
Mar. 15, 2016 (JP) 2016-051218

(51) Int. Cl. A61K 36/185; A61K 9/00; A23L 33/105

(52) U.S. Cl. CPC A61K 36/185; A61K 2236/331; A23L 33/105; A61K 9/0053

(57) ABSTRACT

An object of the present invention is to provide a novel polysaccharide digestion inhibitor of plant origin.

A polysaccharide digestion inhibitor comprising an Astilbe thunbergii extract as an active ingredient.

### RELATED APPLICATIONS

This application claims the priority of Japanese Patent Applications No. 2016-51218 filed on May 15, 2016, which is incorporated herein by reference.

### TECHNICAL FIELD

The present invention relates to a polysaccharide digestion inhibitor comprising an extract of Astilbe thunbergii (Akashoma) as an active ingredient, and more particularly to an α-amylase and α-glucosidase inhibitor comprising an extract of Astilbe thunbergii as an active ingredient.

### BACKGROUND OF THE INVENTION

Carbohydrates are an important energy source, but their excessive ingestion is known to promote obesity and even increase the risk of developing diabetes and the like. However, meals have functioned as an important social role to provide an opportunity for interaction and information exchange among people since ancient times, and it is also an important means of stress release for stressful contemporary people. Therefore, there is a strong need for a method that can substantially exert the same effect as a dietary restriction without impairing such functions of the meal.

Carbohydrates ingested by meals are first decomposed into disaccharides by the action of α-amylase contained in saliva and pancreatic juice. Thereafter, they are decomposed into monosaccharides by the action of α-glucosidase localized on the membrane of the small intestine mucosal epithelial cell, transported into the cell by a transporter localized on the membrane, and then transported into the blood. Since polysaccharides are not taken up into the small intestine mucosal epithelial cell unless they are decomposed into monosaccharides, it is possible to substantially restrict the ingestion of polysaccharides by inhibiting the activity of α-amylase or α-glucosidase.

Until now, a plurality of drugs the action mechanism of which is the inhibition of the activity of α-amylase and/or α-glucosidase have been developed, and some of them have been prescribed to type 2 diabetic patients as agents for ameliorating postprandial hyperglycemia. Representative examples of such a drug include Acarbose (Glucobay, Bayer Yakuhin, Ltd.), Miglitol (Seibule, SANWA KAGAKU KENKYUSHO CO., LTD.), and Voglibose (Basen, Takeda Pharmaceutical Company Limited). However, these drugs are known to have various side effects, and natural plants and their extracts have thus been vigorously analyzed in search of polysaccharide digestion inhibitors with fewer side effects. As extracts that exhibit α-amylase and/or α-glucosidase-inhibitory effect even when orally ingested, an alcohol extract of evening primrose seeds (Patent Literature 1), an ethanol/hydrous ethanol extract of chestnut astringent skins (Patent Literature 2), a hydrous ethanol extract of black rice (Patent Literature 3), a water or (hydrous) organic solvent extract of Rosa rugosa petals and pseudocarps (Patent Literature 4), a water/organic solvent extract of olive leaves (Patent Literature 5) have been reported.

### PRIOR ART DOCUMENTS

### Patent Documents

### [Patent Document 1] WO02/009734

### [Patent Document 2] WO2006/030929

### [Patent Document 3] Japanese Unexamined Patent Application Publication No. 2004-91462

### [Patent Document 4] Japanese Unexamined Patent Application Publication No. 2005-306801

### [Patent Document 5] Japanese Unexamined Patent Application Publication No. 2002-10753

### [Patent Document 6] Japanese Unexamined Patent Application Publication No. 2003-342185

### [Patent Document 7] Japanese Unexamined Patent Application Publication No. 2009-102419

### [Patent Document 8] Japanese Unexamined Patent Application Publication No. 2004-91464

### [Patent Document 9] Japanese Unexamined Patent Application Publication No. 2013-184974

### [Patent Document 10] Japanese Unexamined Patent Application Publication No. 2007-119373

### [Patent Document 11] Japanese Unexamined Patent Application Publication No. 2016-3225

### DISCLOSURE OF THE INVENTION

### Problem to be Solved by the Invention

The present invention has been made in view of the above technical background and is aimed at providing a novel polysaccharide digestion inhibitor of plant origin.

### Means to Solve the Problem

As a result of earnest investigations made for achieving the above objects, the present inventors have found that a hot water or hydrous organic solvent extract of Astilbe thunbergii has a superior α-amylase-inhibiting activity and α-glucosidase-inhibiting activity, and have thus completed the present invention.

Akashoma (scientific name: Astilbe thunbergii var. thunbergii), which is a plant of Saxifragaceae Astilbe, is a perennial herb that grows naturally in mountains of Honshu, Shikoku and Kyushu. Roots of Astilbe thunbergii have been long used as a substitute for Cimicifuga rhizome (roots of Ranunculaceae Cimicifuga simplex) used because of its efficacies such as analgesia, detoxification and anti-inflammation. According to the notification from the Ministry of Health, Labor and Welfare, Cimicifuga rhizome is a medicinal product, but roots of Astilbe thunbergii correspond to a non-medicinal product (see https://hfnet.nih.go.jp/usr/annzenn/image/iyakuhin2).

In recent years, it has been reported that a dry powder of rhizomes of Astilbe thunbergii and its extract (an extract with a water/organic solvent mixture or an organic solvent) has a lipase-inhibiting activity (Patent Literature 6), and that in rats ingesting the dry powder or extract at the same time as a high fat feed, increases in triglyceride and cholesterol levels in blood are suppressed as compared with rats in the control group ingesting only the high fat feed (Patent Literature 7). It has also been shown that in adipocytes of rats orally ingesting the dry powder or extract, lipolysis induced by norepinephrine or adrenocorticotropic hormone (ACTH) is enhanced (Patent Literature 8). Based on these results, obesity inhibitors and blood cholesterol-reducing agents having as active ingredients the dry powder of rhizomes of Astilbe thunbergii and its extract have been proposed (Patent Literatures 7 and 8).

In addition, it has been shown that the dry powder of rhizomes of Astilbe thunbergii and its extract inhibit in vitro the Maillard reaction (one of condensation reactions occurring between a carbonyl group of a reducing sugar and an amino group of an amino compound) occurring between a dextrose and a protein (Patent Literature 9), and that they also have an activity to decompose α-dicarbonyl compound which is one of products resulting from the Maillard reaction (Patent Literature 10).

In addition, regarding the relationship with the blood glucose level control, it has been reported that in adipocytes isolated from rats orally ingesting the dry powder of rhizomes of Astilbe thunbergii or its extract (an extract with a water/organic solvent mixture or an organic solvent), the steatogenesis induced by insulin is markedly decreased (Patent Literature 8). Insulin is a hormone that acts mainly on the liver, the muscle and adipocytes to lower blood glucose levels, and allows adipocytes to promote the uptake of dextrose from the blood and the conversion of dextrose to fat. Therefore, it has been suggested that the dry powder of rhizomes of Astilbe thunbergii or its extract have an activity of inhibiting the action of insulin on adipocytes. It has been reported that a hot water extract of roots of Astilbe thunbergii has an activity of inhibiting Dipeptidyl peptidase-4 (hereinafter abbreviated as DPP-IV) which is known to decompose gastrointestinal hormone (incretin) promoting insulin secretion (Patent Literature 11), and the possibility that the secretion amount of insulin may increase by ingesting the extract has also been suggested.

Thus, it has been known that the extract of roots or rhizomes of Astilbe thunbergii can inhibit the digestion and absorption of fat and have an effect on the secretion amount of insulin and its action, but there has been no report suggesting that it has a role in the digestion of polysaccharides.

Accordingly, the present invention includes the following:

[1] A polysaccharide digestion inhibitor comprising an Astilbe thunbergii extract as an active ingredient;
[2] The polysaccharide digestion inhibitor according to the [1], wherein the inhibitor is α-amylase inhibitor and/or α-glucosidase inhibitor;
[3] The polysaccharide digestion inhibitor according to the [1] or [2], wherein the extract is an extract with water and/or an organic solvent;
[4] A food and drink comprising the polysaccharide digestion inhibitor according to any one of the [1] to [3]; and
[5] An oral composition comprising the polysaccharide digestion inhibitor according to any one of the [1] to [3].

### Effect of the Invention

According to the present invention, an α-amylase and α-glucosidase inhibitor derived from Astilbe thunbergii is provided. Oral ingestion of the inhibitor allows substantial intake restriction of polysaccharides.

### BRIEF DESCRIPTION OF THE DRAWINGS

The plot or bar in the Figures represents the mean value of the measurements obtained for 7 GK rats per experimental group, and the error bar represents its standard deviation. The asterisk (*) and double asterisk (**) in the Figures indicate that as a result of the test of significance, a statistical significance between a negative control group and the other group was confirmed with p value<0.05, p value<0.01, respectively.

FIG. 1 is a graph showing time-dependent changes in the blood glucose levels in GK rats orally ingesting starch after orally ingesting an Astilbe thunbergii extract.

FIG. 2 is a graph showing the area under the blood concentration-time curve of glucose (hereinafter abbreviated as glucose AUC) in GK rats orally ingesting starch after orally ingesting an Astilbe thunbergii extract.

FIG. 3 is a graph showing time-dependent changes in the blood glucose levels in GK rats orally ingesting sucrose after orally ingesting an Astilbe thunbergii extract.

FIG. 4 is a graph showing the glucose AUC in GK rats orally ingesting sucrose after orally ingesting an Astilbe thunbergii extract.

FIG. 5 is a graph showing time-dependent changes in the blood glucose levels in GK rats orally ingesting dextrose after orally ingesting an Astilbe thunbergii extract.

FIG. 6 is a graph showing the glucose AUC in GK rats orally ingesting dextrose after orally ingesting an Astilbe thunbergii extract.

### DESCRIPTION OF THE PREFERRED EMBODIMENT

Preferred embodiments of the present invention are described below.

According to the present invention, a polysaccharide digestion inhibitor comprising as an active ingredient an Astilbe thunbergii extract having an α-amylase and/or α-glucosidase-inhibiting activity is provided. “Polysaccharide” used in the present invention refers to “two or more monosaccharides polymerized by a glycosidic bond (preferably, α-1,4-glycosidic bond)”.

In the present specification, “dextrose” is sometimes referred to as “glucose” according to the practice in the art, but there is no difference in meaning between the two. In the present specification, “%” means “% by mass” unless otherwise specified.

<Astilbe thunbergii Extract>

Akashoma (Astilbe thunbergii var. thunbergii) used for the present invention may be either naturally growing or cultivated Astilbe thunbergii, and its preferred parts are roots and/or rhizomes. Both of raw and dried plants can be used, but from the viewpoint of improved extraction efficiency, it is preferable to be chopped or powdered.

The solvent to be used for extracting Astilbe thunbergii may be any of water, an organic solvent, a mixture of water and an organic solvent (hereinafter sometimes referred to as a hydrous organic solvent), or it may be subjected to stepwise extraction with the combination of these solvents. The term “stepwise extraction” used herein refers to further extraction of the extract obtained by a solvent with another solvent.

Among the solvents, water, a hydrous organic solvent, or a combination thereof (specifically, extraction with a hydrous organic solvent followed by extraction with water) is preferably used. The water content of the hydrous organic solvent is 10 to 90 v/v %, preferably 20 to 80 v/v %, more preferably 30 to 70 v/v %, and most preferably 40 to 60 v/v %.

Examples of the organic solvent include, but not particularly limited to, a lower alcohol such as methanol, ethanol, propanol or butanol; an ester such as ethyl acetate; a glycol such as ethylene glycol, butylene glycol, propylene glycol, 1,3-butylene glycol or glycerin; an ether such as diethyl ether or petroleum ether; a polar solvent such as acetone or acetic acid; or a hydrocarbon such as benzene, hexane or xylene. Among them, a lower alcohol and an ester are preferred, and a lower alcohol is more preferred. The organic solvent may be used alone or in combination of two or more.

The extraction temperature can be appropriately adjusted in the range from an ordinary temperature to the boiling point of the solvent depending on the type of solvent to be used. The extraction may be carried out under pressure, normal pressure or reduced pressure. The extraction time may also be appropriately adjusted depending on the type of solvent to be used. For example, in the case of using water as an extraction solvent, the extraction may be carried out at the temperature in the range of 20 to 140° C., preferably 60 to 130° C. and more preferably 80 to 125° C. within a period ranging from 1 minute to 1 hour and preferably 10 to 30 minutes. In the case of using hydrous ethanol as an extraction solvent, the extraction may be carried out at the temperature in the range of 20 to 100° C. and preferably 40 to 80° C. within a period ranging from 1 minute to 1 hour and preferably 10 to 30 minutes. In addition, it is more preferable to stir or reflux the solvent during the heat retention period.

The amount of solvent to be used for the extraction is not particularly limited, and the solvent may be used in the amount (mass ratio) of 2 to 50 times, preferably 5 to 50 times and more preferably 10 to 30 times the dry matter of roots and/or rhizomes of Astilbe thunbergii.

After the extraction, solids can be removed by filtration, centrifugation or the like to obtain an extraction liquid of Astilbe thunbergii. The extraction liquid of Astilbe thunbergii may be used as it is as an Astilbe thunbergii extract, or may be used as an Astilbe thunbergii extract after concentrating it, or drying and solidifying it, or removing the solvent therefrom. Removal of the solvent may be carried out by any method well known to those skilled in the art, for example, by distilling off the solvent under reduced pressure, or freeze drying, or the like.

The Astilbe thunbergii extract to be used may also be a commercially available product. For example, “Astilbe thunbergii extract powder” (manufactured by BHN Co., Ltd.), which is a hydrous ethanol extract of dried Astilbe thunbergii roots, may be used.

The Astilbe thunbergii extract may be further subjected to a conventional purification method to highly purify it, and the obtained purified product may be used as an active ingredient of a polysaccharide digestion inhibitor according to the present invention. Examples of the purification method includes adsorption and desorption using activated carbon, silica gel, a polymer-based carrier or the like; column chromatography; liquid-liquid extraction; fractional precipitation; or any combination thereof.

<Intended Use and Usage>

The Astilbe thunbergii extract obtained by the above method can function as an α-amylase inhibitor, or an α-amylase and α-glucosidase inhibitor. Therefore, a polysaccharide digestion inhibitor is provided by blending the Astilbe thunbergii extract.

In the present invention, the term “α-amylase inhibitor” refers to a substance which inhibits α-1,4-glycosidic bond cleavage activity of α-Amylase (EC 3.2.1.1) when ingested orally (i.e., in the digestive organs of animals). The α-amylase-inhibiting activity can be evaluated, for example, as an activity to significantly decrease the amount of dextrose produced from starch orally ingested by an animal (it may be evaluated as blood glucose level).

In the present invention, the term “α-glucosidase inhibitor” refers to a substance which inhibits α-1,4-glycosidic bond cleavage activity of α-glucosidase (EC3.2.1.20) when ingested orally (i.e., in the digestive organs of animals). The inhibiting activity can be evaluated, for example, as an activity to significantly decrease the amount of dextrose produced from sucrose orally ingested by an animal (it may be evaluated as blood glucose level).

The polysaccharide digestion inhibitor according to the present invention may comprise 0.01 to 100% and preferably 0.1 to 100% of the Astilbe thunbergii extract (in terms of the extract in the case of a product further purified from the extract).

The polysaccharide digestion inhibitor according to the present invention can inhibit the decomposition of a polysaccharide into monosaccharides by inhibiting the activity of α-amylase and/or α-glucosidase. Therefore, the polysaccharide digestion inhibitor according to the present invention can be suitably used for intake restriction of carbohydrates, suppression of an increase in blood glucose after meals, and/or amelioration of postprandial hyperglycemia caused by impaired glucose tolerance.

Since the polysaccharide digestion inhibitor according to the present invention does not substantially inhibit the absorption of monosaccharides, it is thought that a hypoglycemic state may be less likely to be caused extremely low even if it is ingested excessively. In addition, since it does not substantially inhibit the mechanism for normalizing postprandial blood glucose, healthy individuals having no abnormality in glucose tolerance can also be safely ingested.

The polysaccharide digestion inhibitor according to the present invention is preferably ingested orally before ingestion of polysaccharides (before meal) or during meal, but it is thought that if it is ingested shortly after meal, the polysaccharide digesting agent can fully exert its effect by oral ingestion. Therefore, it is ingested from 30 minutes before meal to 30 minutes after meal, preferably from 15 minutes before meal to 15 minutes after meal, more preferably from 10 minutes before meal to 10 minutes after meal and most preferably from 5 minutes before meal to during meal.

The ingestion amount (amount of polysaccharides) can be adjusted depending on the contents of a meal, and in the case of a meal of general contents is as a rough indication, for example, 50 to 2500 mg/meal, preferably 125 to 2000 mg/meal, more preferably 250 to 1500 mg/meal, and most preferably 500 to 1250 mg/meal, based on an average body weight of 60 kg (human), in terms of the amount of crude drugs of Astilbe thunbergii root. In the case of a hydrous ethanol (e.g., an ethanol concentration of 50 to 60 v/v %) extract of Astilbe thunbergii root, the ingestion amount may be, for example, 10 to 500 mg/meal, preferably 25 to 400 mg/meal, more preferably 50 to 300 mg/meal and most preferably 100 to 250 mg/meal in terms of a dry weight of the extract. As described above, it is thought that the polysaccharide digestion inhibitor according to the present invention is at low risk for causing a hypoglycemic state even if it is ingested excessively, but it is preferably ingested as a rough indication in the amount not more than 3000 mg/day (in terms of the amount of crude drugs of Astilbe thunbergii root).

The polysaccharide digestion inhibitor according to the present invention may be ingested alone or may be in the form of an oral composition comprising a pharmaceutically acceptable carrier, excipient, plasticizer, coloring agent, preservative or the like. Examples of the carrier to be used in the oral composition include a sugar alcohol (such as mannitol), an inorganic substance (such as calcium carbonate), microcrystalline cellulose, a cellulose (such as carboxymethylcellulose), gelatin, sodium alginate, polyvinylpyrrolidone, agar, magnesium stearate or talc. Although a disaccharide (such as lactose) and a polysaccharide (such as starch or corn starch) are carriers widely used in the art, they are substrates for α-amylase and/or α-glucosidase, and it is therefore preferable in the present invention that these carriers are not used in large amounts.

The form of the oral composition is not particularly limited, but it may be in the form of a tablet, a pill, a capsule, a granule, a powder, a troche, a solution (beverage), or the like.

The polysaccharide digestion inhibitor according to the present invention can also be suitably ingested in the form of a common food, a health food, a health functional food (food for specified health uses, food with functional claims, etc.) comprising it.

Examples of the food include a beverage such as a milk drink, a lactic acid bacteria beverage, a soft drink, a carbonated drink, a fruit juice drink, a vegetable drink, an alcoholic beverage, a powdered drink, a coffee beverage, a tea beverage, a green tea beverage or a barley tea beverage; a confectionery such as a pudding, a jelly, a Bavarian cream, a yogurt, an ice cream, a gum, a chocolate, a candy, a caramel, a biscuit, a cookie or a rice cracker such as okaki or senbei; a soup such as a consomme or a potage soup; various seasonings such as miso, a soy sauce, a dressing, a ketchup, a baste, a sauce or a Furikake (dried food sprinkled over rice); a jam such as a strawberry jam, a blueberry jam, a marmalade or an apple jam; a fruit liquor such as a red wine; a fruit for processing such as a cherry, an apricot, an apple, a strawberry or a peach preserved in a syrup; a noodle such as udon, hiyamugi, somen, buckwheat noodle, Chinese noodle, spaghetti, macaroni, rice vermicelli, harusame (bean-starch vermicelli) or vermicelli or wonton wrapper; or various processed foods.

The polysaccharide digestion inhibitor according to the present invention can exert the above effects not only for a human but also for animals other than a human. Therefore, the polysaccharide digestion inhibitor according to the present invention can also be blended in a feedstuff for a farm or companion animal.

### EXAMPLES

Hereinafter, the present invention will be described more specifically with reference to Examples. However, the present invention is not intended to be limited by these examples by any means.

### Test Example 1: Screening for Library of Materials

The present inventors have created a library of materials (294 materials) consisting of freeze-dried products and extract powders of plants, obtained mainly on a commercial basis in Japan and from abroad, and they have found 51 materials having a DPP-IV-inhibiting activity by screening the library (Patent Literature 11). Accordingly, a hot water extract was prepared from each material (Approach 1), and the α-amylase inhibiting activity was analyzed by subjecting it to the test described in Approach 2.

[Approach 1: Preparation of Hot Water Extract]

Each material was suspended in ultrapure water (MilliQ water) in the amount of 10 times the material and extracted with an autoclave at 121° C. for 15 minutes. Thereafter, it was subjected to centrifugation (at 10,000 rpm for 10 minutes), and the supernatant was recovered and used as a hot water extract.

In the following Approaches 2 and 3, the hot water extract diluted 100-fold with a 50% DMSO aqueous solution was used as a test substance.

[Approach 2: In Vitro Amylase-Inhibiting Activity Test]

Reaction Solution (500 μl)

(1) Test substance in a 50% DMSO aqueous solution, 100 μl

(2) Starch azure in buffer, 350 μl

(3) 0.5 U/mL Porcine pancreas-derived-α-amylase in buffer, 50 μl
buffer: 0.1 M Tris-hydrochloric acid buffer containing 0.01 M CaCl₂ (pH 6.9)

Procedure

The (2) was placed in a 2.0 ml microtube and incubated at 37° C. for 5 minutes, followed by addition in the order of the (1) and the (3) and further incubation at 37° C. for 15 minutes with shaking. The reaction was stopped by adding 50% acetic acid followed by centrifugation (at 4° C. and 1500×g for 5 minutes), and 200 μl of the supernatant was transferred to a 96-well microplate and the absorbance at 595 nm was measured. The buffer was used instead of the enzyme solution in a blank, a 50% DMSO aqueous solution was used instead of the test substance in a negative control, and acarbose (10 μM) was used as a test substance in a positive control.

Duplicate measurements were carried out for each test substance, the average value was calculated, and the α-amylase activity inhibition rate was calculated according to the following formula (1). In the following formula (1), ODₛₐₘₚₗₑ represents the absorbance of a well to which the test substance was added, OD_{sample blank} represents the absorbance of a well to which the buffer was added instead of the enzyme in the presence of the test substance, OD_{control} represents the absorbance of a well of the negative control, and OD_{control blank} represents the absorbance of a well to which the buffer is added instead of the enzyme in a negative control.

$$\begin{matrix}[ Mathematical 1 ] & \\ \begin{matrix}\begin{matrix}Inhibition rate \\ of enzyme \\ activity ( \% )\end{matrix} = \{ 1 - \frac{( {OD}_{sample} - {OD}_{sample blank} )}{( {OD}_{control} - {OD}_{control blank} )} \} \times 100 & \end{matrix} & ( 1 )\end{matrix}$$

Among the above 294 materials, only 15 materials inhibited strongly (specifically, by 80% or more) the activity of α-amylase, and 229 materials (77.9%) had an α-amylase activity inhibition rate of 50% or less. Interestingly, among 51 materials a DPP-IV-inhibiting activity of which had been confirmed in Patent Literature 11, 42 materials (rose, Chaga, Chinese quince, Lycii fructus, Sasa veitchii, Cassia seed, ginseng, Crataegus cuneata, Alpinia speciosa lobe, Tian Cha, Eucommia ulmoides tea, silvervine, pinecone, unpolished Oryza sativa, Chrysanthemum flower, Averrhoa carambola leaf, Jujube, Astragalus complanatus R.Br., Rosa roxburghii, Linden, yeast peptide, broccoli sprout, green szechuan pepper, Roselle (roselle calyx), Dianthus caryophyllus, Ginkgo biloba leaf, Eleutherococci senticosi rhizoma, German chamomile, black garlic, Houttuynia cordata, lotus embryo, Piper longum, Monascus purpureus, Acer maximowiczianum, Smallanthus sonchifolius leaf, Artemisia princeps, Siraitia grosvenorii, Ganoderma lucidum, Melissa officinalis, black vinegar, Camellia japonica seed, Canarium album and torch rose) had an α-amylase activity inhibition rate of 50% or less. Furthermore, among the above 42 materials, no α-amylase-inhibiting activity was substantially detected in 39 materials except for rose, Ginkgo biloba and Linden.

The present inventors analyzed the 15 materials exhibiting an α-amylase activity inhibition rate of 80% or more for the α-glucosidase-inhibiting activity according to the following Approach 3.

[Approach 3: In Vitro Glucosidase (Maltase)-Inhibiting Activity Test]

Reaction Solution (500 μl)

(1) Test substance in demineralized water, 100 μl

(2) 3.5 mM maltose in 0.1M phosphate buffer (pH 6.3), 350 μl

(3) Crude enzyme solution prepared from small intestinal acetone powder from rat (Sigma), 350 μl

Procedure

The (1) and (2) were placed in a 2.0 ml microtube and incubated at 37° C. for 5 minutes, followed by addition of the (3) and further incubation at 37° C. for 15 minutes with shaking. The reaction was stopped by adding 750 μl of a 2 M Tris-HCl buffer (pH 7.0), the reaction mixture was then passed through a reversed-phase short column, and the amount of free glucose was determined using Glucose CII-Test Wako (Wako Pure Chemical Industries, Ltd.).

Demineralized water was used instead of the test substance in a negative control, and acarbose (5 μM) was used as a test substance in a positive control. In addition, a value obtained from a solution obtained by addition of a reaction stop solution (a 2M Tris-HCl buffer (pH 7.0), 750 μl) to the reaction solution followed by incubation (at 37° C. for 15 minutes) was subtracted from each measurement value as a background derived from the sample. The α-glucosidase activity inhibition rate of each test substance was expressed as an inhibition rate against the α-glucosidase activity of the negative control.

As a result of the above analysis, Astilbe thunbergii was found to be a material having a remarkable α-amylase-inhibiting activity (inhibition rate of 86%) and also having an α-glucosidase-inhibiting activity (inhibition rate of approximately 20%).

### Test Example 2: In Vitro Enzyme Activity Inhibitory Action of Astilbe thunbergii Extract

The Astilbe thunbergii material used in the Approach 1 was a dry powder (i.e., a hydrous ethanol extract) obtained by extracting Astilbe thunbergii root with hydrous ethanol (an ethanol concentration of 50 to 60 v/v %), filtering it to remove residues and then spray drying it (“Astilbe thunbergii extract powder” manufactured by BHN Co., Ltd., its ratio to the crude drug: 5:1). Accordingly, the hydrous ethanol extract was analyzed for an α-amylase-inhibiting activity (Approach 2), an α-glucosidase-inhibiting activity (Approach 3) and a DPP-IV inhibiting activity (using DPP-4 inhibitor screening assay kit (manufactured by Cayman Chemical Company; see Patent Literature 11). The results are shown in Table 1.

TABLE 1

| | Concentration of Astilbe | | | | |
|---|---|---|---|---|---|
| | thunbergii extract (mg/ml) | | | | |
| Inhibition rate of enzyme activity | 1.0 | 0.5 | 0.2 | 0.1 | 0.01 |
| Inhibition rate of α-amylase activity | 93 | ND | 97 | ≥95 | ≤10 |
| Inhibition rate of α-glucosidase activity | 20 | ND | ND | 4 | ND |
| Inhibition rate of DPP-IV activity | 36 | 2 | ND | ND | ND |

ND: Not determined

As shown in Table 1, the hydrous ethanol extract of Astilbe thunbergii inhibited the α-amylase activity by 95% or more at a final concentration in the range of 0.1 mg/ml to 1 mg/ml. Since the inhibition rate decreased to several % at 0.01 mg/ml, IC₅₀ (a 50% inhibition concentration) of an α-amylase-inhibiting activity of the extract is estimated to be between 0.01 mg/ml and 0.1 mg/ml. Since the extract inhibited an α-glucosidase activity by 20% and a DPP-IV activity by 36% at a final concentration of 0.1 mg/ml, the Astilbe thunbergii extract is shown to also have inhibiting activities against α-glucosidase and DPP-IV.

It was therefore revealed that the hot water and hydrous organic solvent extracts have α-amylase-, α-glucosidase- and DPP-IV-inhibiting activities, and that among them the α-amylase-inhibiting activity is most remarkable.

### Test Example 3: In Vivo Starch Digestion Inhibitory Action of Astilbe thunbergii Extract

The analysis was carried out for whether α-amylase- and α-glucosidase-inhibiting activities found in the Astilbe thunbergii extracts were exhibited even when it was orally ingested.

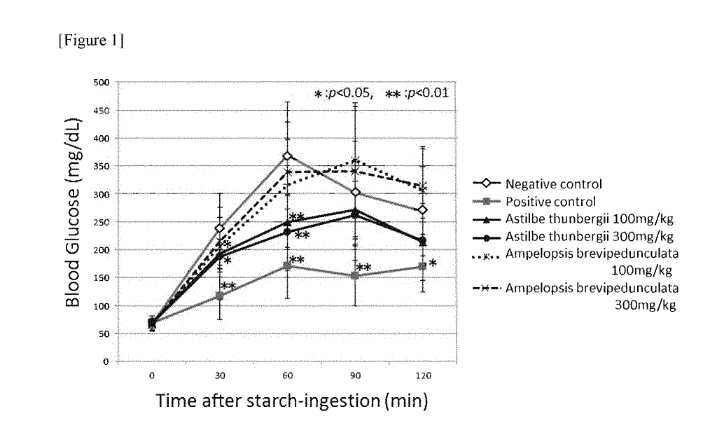

Each GK rat (Goto-Kakizaki rat) was allowed to orally ingest an Astilbe thunbergii extract and immediately thereafter to orally ingest starch, and the blood glucose level up to 2 hours later was measured over time (Approach 4). In the case of a GK rat, since hyperglycemia is caused due to its low insulin secretion amount after meal, it is possible to sensitively analyze the influence of drugs on the digestion and absorption of saccharides. For comparison, in the screening of Test Example 1, a hot water extract of Ampelopsis brevipedunculata var. heterophylla which had been found to be a material having an α-amylase-inhibiting activity (α-amylase-inhibiting activity: 71%) was also analyzed in the same manner as for Astilbe thunbergii. Analysis results are shown in FIG. 1.

[Approach 4: Measurement of Blood Glucose Level after Loading Starch]

Conditioning GK Rats

GK rats (male, 5 weeks old) were obtained from Japan SLC, Inc. and preliminarily bred for 1 week before use. Seven rats per experimental group were used.

Test Substance

(1) Japanese Pharmacopeia water for injection (a negative control))

(2) A solution having acarbose dissolved in the water for injection (a positive control); administered at 10 mg/kg body weight

(3) A suspension having the hydrous ethanol extract of Astilbe thunbergii suspended in the water for injection; administered at 100 mg/kg body weight or 300 mg/kg body weight

(4) A suspension having a hot water extract of Ampelopsis brevipedunculata var. heterophylla suspended in the water for injection; administered at 100 mg/kg body weight or 300 mg/kg body weight

Administration and Measurement of Blood Glucose Level

Each GK rat was fasted for 18 hours and its fasting blood glucose level was then measured. Thereafter, the test substance was orally administered to the rat using a disposable gastric probe and a disposable syringe. Two minutes after the administration of the test substance, starch (dissolved in purified water, at 2 g/kg body weight) was administered in a single oral dose. Blood was collected from the tail vein 30, 60, 90 and 120 minutes after the administration of starch, and the blood glucose level was measured using a simple type blood glucose meter (NIPRO Freestyle Freedom Lite, FS Blood Glucose Sensor Light, manufactured by Nipro Corporation).

As shown in FIG. 1, in the negative control, the blood glucose level increased immediately after ingesting starch, and reached a maximum value (396±96 mg/dL) 60 minutes later, and thereafter turned to a decrease. By contrast, in the positive control, the blood glucose level increased very slowly, and remain as low as 117±43 mg/dL even 60 minutes later. In the positive control, it was thought that the digestion of starch was inhibited (that is, the amount of dextrose produced decreased) due to the α-amylase and α-glucosidase activities inhibited by acarbose, and the blood glucose level thus increased slowly.

Also in the experimental group ingesting the Astilbe thunbergii extract according to the present invention, the blood glucose level was slowly increased. In the case of ingesting both doses of 100 mg/kg and 300 mg/kg, the blood glucose levels after 30 minutes and 60 minutes were significantly lower than those of the negative control (blood glucose level after 60 minutes: 250±26 mg/dL (100 mg/kg body weight), 231±27 mg/dL (300 mg/kg body weight)). Therefore, oral ingestion of the Astilbe thunbergii extract according to the present invention was shown to inhibit the digestion of starch and lead to slowing down the increase in blood glucose level.

By contrast, in the experimental group ingesting the extract of Ampelopsis brevipedunculata var. heterophylla, the blood glucose level had no significant difference from that in the negative control, at any measurement time from the ingestion of starch to 2 hours later. Therefore, it was revealed that the oral ingestion of the extract of Ampelopsis brevipedunculata var. heterophylla can exhibit substantially no inhibitory action against α-amylase and α-glucosidase. This may be because an ingredient responsible for the inhibiting activity contained in the extract of Ampelopsis brevipedunculata var. heterophylla may have been decomposed by digestive enzymes.

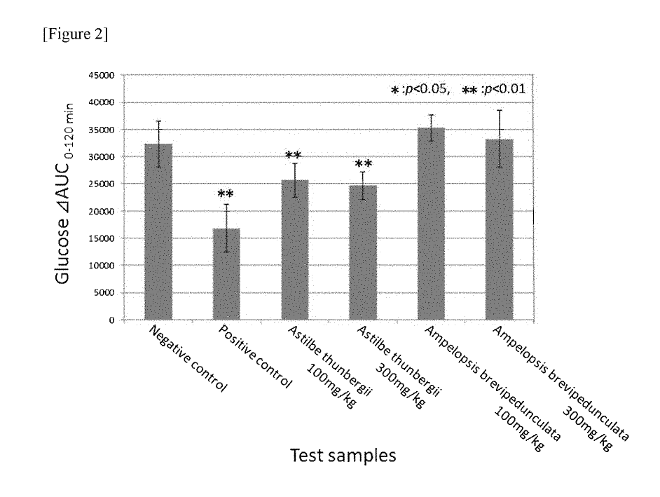

FIG. 2 and Table 2 show the glucose AUC (Area under the blood glucose curve) from 0 to 120 minutes after ingesting starch (AUC_{0-12 min}). In the experimental group ingesting the Astilbe thunbergii extract, it is shown that the glucose AUC was greatly decreased to approximately 79% (at 100 mg/kg body weight) and 76% (at 300 mg/kg body weight) of the negative control.

TABLE 2

| | Glucose ΔAUC_{0-120 min} after loading starch or sugar | | |
|---|---|---|---|
| | Mean ± SD, n = 7 (in terms of %) | | |
| Test substance | Starch | Sucrose | Glucose |
| Negative control | 32327 ± 4244 | 24731 ± 2850 | 33028 ± 3552 |
| | (100 ± 13) | (100 ± 12) | (100 ± 11) |
| Positive control | 16811 ± 4389** | 19074 ± 2177** | 25277 ± 3285** |
| | (52 ± 14) | (77 ± 9) | (77 ± 10) |
| Astilbe thunbergii, | 25639 ± 3121** | 22247 ± 3480 | 30435 ± 3259 |
| 100 mg/kg body weight | (79 ± 10) | (90 ± 14) | (92 ± 10) |
| Astilbe thunbergii, | 24686 ± 2546** | 20631 ± 2109** | 30684 ± 4413 |
| 300 mg/kg body weight | (76 ± 8) | (83 ± 9) | (93 ± 13) |
| Ampelopsis brevipedunculata, | 35284 ± 2367 | 28682 ± 3388 | ND |
| 100 mg/kg body weight | (109 ± 7) | (116 ± 14) | |
| Ampelopsis brevipedunculata, | 33272 ± 5221 | 31427 ± 5338 | ND |
| 300 mg/kg body weight | (103 ± 16) | (127 ± 22) | |

*p < 0.05,
**p < 0.01,
ND: Not determined

The above results suggested that the Astilbe thunbergii extract according to the present invention could inhibit the α-amylase and/or α-glucosidase activities in the body of an animal even when ingested orally and it could significantly inhibit the digestion of polysaccharides. Accordingly, it was revealed that oral ingestion of the Astilbe thunbergii extract at the same time as or slightly before oral ingestion of polysaccharides decreased the amount of dextrose which transferred to the blood decreases (as compared with in the case of no ingestion) and increased the blood glucose level slowly.

### Test Example 4: Inhibitory Effect of Astilbe thunbergii Extract on In Vivo Sucrose Digestion

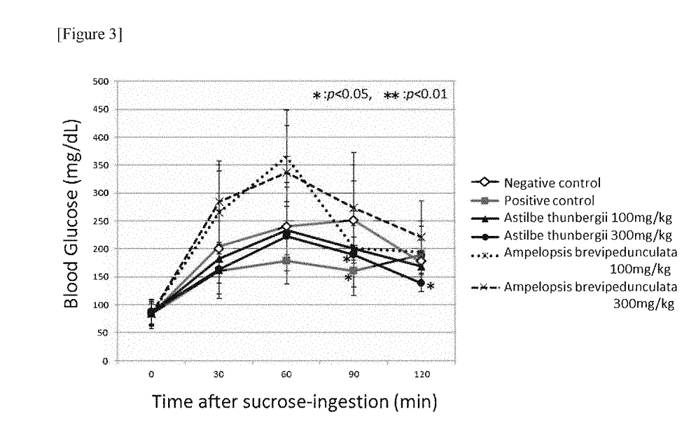

As described above, since the digestion process from starch to dextrose depends on the enzyme activities of both α-amylase and α-glucosidase, it cannot be ruled out from the results of Test Example 3 that the Astilbe thunbergii extract might inhibit only one of the enzyme activities in the body of an animal (the activity of the other enzyme activity cannot be inhibited). Accordingly, the digestion inhibitory effect of Astilbe thunbergii extract was analyzed using sucrose for which the digestion process depends only on α-glucosidase. Specifically, in the Approach 4, the blood glucose was measured using “sucrose (dissolved in purified water, at 2 g/kg body weight)” instead of “starch (dissolved in purified water, at 2 g/kg body weight)”. In this analysis, 7-week-old male GK rats were used (7 rats/experimental group). Results are shown in FIG. 3.

As shown in FIG. 3, in the negative control, the blood glucose level increased immediately after ingesting starch, and reached a maximum value (251±71 mg/dL) 90 minutes later, and thereafter turned to a decrease. By contrast, in the positive control, the increase in the blood glucose level was small, and remained as low as 161±45 mg/dL even 90 minutes later. In the positive control, it was thought that the amount of dextrose produced from sucrose decreased due to the α-glucosidase activity inhibited by acarbose, and the increase in the blood glucose level thus decreased.

Also in the experimental group ingesting the Astilbe thunbergii extract according to the present invention, the increase in the blood glucose level was smaller than that in the negative control. In the experimental group to which the Astilbe thunbergii extract was administered at 100 mg/kg body weight, there was no significant difference in the blood glucose level from the negative control. By contrast, in the experimental group to which the Astilbe thunbergii extract was administered at 300 mg/kg body weight, the blood glucose levels at 90 minutes and 120 minutes later were significantly lower than those in the negative control (90 minutes later: 189±32 mg/dL, 120 minutes later: 139±15 mg/dL). Therefore, it was shown that the Astilbe thunbergii extract according to the present invention can inhibit the digestion of sucrose in the body of the animal when orally ingested, that is, that it can inhibit the activity of α-glucosidase.

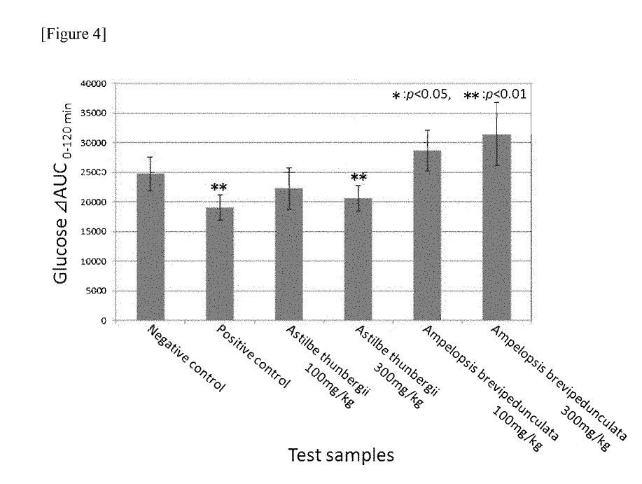

FIG. 4 and Table 2 show the glucose AUC from 0 to 120 minutes after ingesting sucrose. It is shown that in the GK rats ingesting the Astilbe thunbergii extract at 100 mg/kg body weight, there was no significant difference in the glucose AUC from the negative control, but that in the GK rats ingesting the Astilbe thunbergii extract at 300 mg/kg body weight, the glucose AUC significantly decreased to approximately 83% of that in the negative control.

In the case of ingesting the Astilbe thunbergii extract at 100 mg/kg body weight, the increase in the blood glucose level after ingesting starch was significantly inhibited (FIGS. 1 and 2). Since the ca-glucosidase activity was not substantially inhibited in this ingestion amount of the Astilbe thunbergii extract (FIGS. 3 and 4), it is thought that the increase in the blood glucose level after ingesting starch was inhibited at this ingestion amount of the Astilbe thunbergii extract due to the inhibition of the α-amylase activity.

The above results suggested that the Astilbe thunbergii extract according to the present invention could inhibit both the α-amylase and α-glucosidase activities when ingested orally.

### Test Example 5: Influence of Astilbe thunbergii Extract on In Vivo Dextrose Absorption

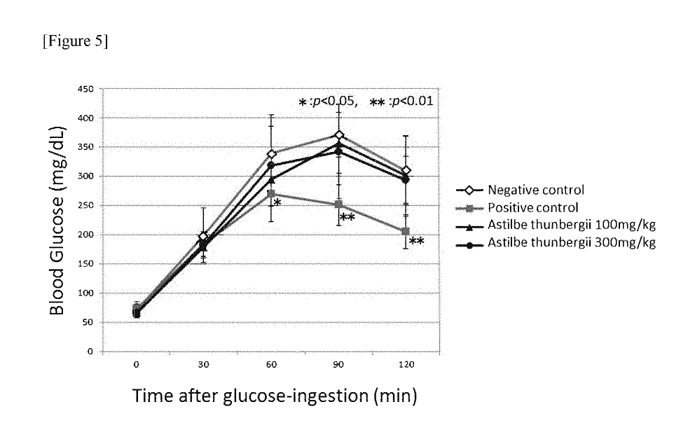

Next, it was confirmed that the Astilbe thunbergii extract according to the present invention did not influence the absorption of dextrose in the small intestine and its subsequent transfer to the blood. Specifically, each GK rat was allowed to orally ingest the Astilbe thunbergii extract and dextrose and its influence on a change in the blood glucose level was analyzed (Approach 5). In this analysis, an experiment group orally ingesting anagliptin, a DPP-IV inhibitor, was used as a positive control. The results are shown in FIG. 5.

[Approach 5: Measurement of Blood Glucose Level after Loading Glucose]

Test Substance

(1) Japanese Pharmacopeia water for injection (a negative control)

(2) A solution having anagliptin dissolved in the water for injection (a positive control); administered at 10 mg/kg body weight

(3) A suspension having, the hydrous ethanol extract of Astilbe thunbergii prepared in the Test Example 2, suspended in the water for injection; administered at 100 mg/kg body weight or 300 mg/kg body weight

Administration and Measurement of Blood Glucose Level

Each GK rat (8-week-old male rat, 7 rats/experimental group) was fasted for 18 hours and its fasting blood glucose level was then measured. Thereafter, the test substance was orally administered to the rat using a disposable gastric probe and a disposable syringe. 15 minutes after the administration of the test substance, dextrose (dissolved in purified water, at 2 g/kg body weight) was administered in a single oral dose. Blood was collected from the tail vein 30, 60, 90 and 120 minutes after the administration of dextrose, and the blood glucose level was measured using a simple type blood glucose meter (NIPRO Freestyle Freedom Lite, FS Blood Glucose Sensor Light, manufactured by Nipro Corporation).

As shown in FIG. 5, in the negative control, the blood glucose level increased immediately after ingesting dextrose, and turned to a decrease since 90 minutes later. The main reason for the decrease in the blood glucose level may be because the consumption of dextrose (conversion to glycogen) in the liver and the uptake of dextrose in the insulin sensitive cells (mainly myocytes and adipocytes) were enhanced by the action of insulin secreted in the blood.

In the experimental group ingesting the Astilbe thunbergii extract according to the present invention, the blood glucose level increased after ingesting dextrose at virtually the same rate as that in the negative control, and the blood glucose level decreased in virtually the same time course as that in the negative control (at any measurement times, no significant difference from that in the negative control).

Therefore, it was shown that the oral ingestion of the Astilbe thunbergii extract according to the present invention did not influence the process from the absorption of dextrose in the small intestine to its transfer to the blood, and the process of the subsequent consumption of (high concentration) dextrose.

As described above, it had been reported that the dry powder of Astilbe thunbergii and its extract have the effect of lowering the insulin sensitivity of adipocytes (Patent Literature 8), but they was confirmed not to lead to a change in the blood glucose level.

On the other hand, in the positive control ingesting anagliptin, a DPP-IV inhibitor, the blood glucose level increased similarly to the negative control after ingesting dextrose, but began to decrease earlier than that in the negative control (specifically, since 60 minutes later). This may be because the activity of DPP-IV was inhibited by anagliptin to delay the decomposition of incretin, so that incretin acted on the pancreas for a long time and the secretion amount of insulin increased.

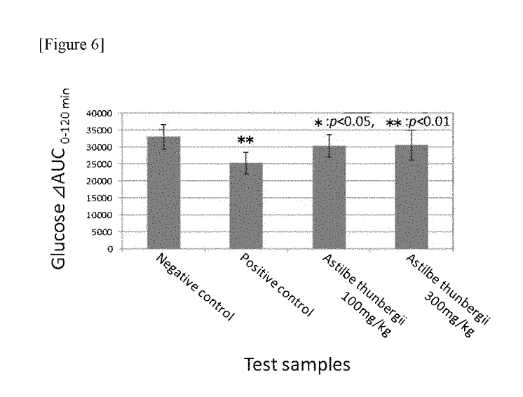

FIG. 6 and Table 2 show the glucose AUC from 0 to 120 minutes after ingesting dextrose. It is shown that the ingestion of anagliptin significantly decreases the glucose AUC, but the ingestion of the Astilbe thunbergii extract dose not significantly change the glucose AUC.

From the above, it was shown that the oral ingestion of the Astilbe thunbergii extract according to the present invention did not substantially inhibit the absorption of a monosaccharide and its transfer to the blood. It was also shown that the Astilbe thunbergii extract according to the present invention did not substantially inhibit the mechanism for normalizing postprandial blood glucose.

From the results of Test Examples 4 to 6, it was revealed that the oral ingestion of the Astilbe thunbergii extract according to the present invention in combination with a polysaccharide could inhibit the substantial ingestion of the polysaccharide.

Examples for a food and drink and an oral composition comprising the Astilbe thunbergii extract according to the present invention are listed below, but the present invention is not limited thereto. Each of the following blending amounts is expressed in % by mass.

### Example 1: Vegetable Juice

### <Formula>

| Ingredient | | Blending amount |
|---|---|---|
| (1) | Hot water extract of Astilbe thunbergii | 0.5 |
| | (prepared in Test Example 1) | |
| (2) | Vegetable squeeze | 84.5 |
| (3) | 5-Times concentrated apple juice | 5.0 |
| (4) | 3-Times concentrated lemon juice | 2.0 |
| (5) | Sodium ascorbate | 0.05 |
| (6) | Purified water | Balance |

| | |
|---|---|
| Total | 100.0 |

### <Preparation Method>

The ingredients (1) to (6) were mixed to obtain a vegetable juice.

### Example 2: Cookies

### <Formula>

| Ingredient | | Blending amount |
|---|---|---|
| (1) | Hydrous ethanol extract of Astilbe | 10.0 |
| | thunbergii prepared in Test | |
| | Example 2) | |
| (2) | Shortening | 40.0 |
| (3) | Cow's milk | 2.0 |
| (4) | Artificial sweetener (aspartame) | 7.5 |
| (5) | Egg | 7.5 |
| (6) | Baking powder | 0.001 |
| (7) | Soft wheat flour | Balance |

| | |
|---|---|
| Total | 100.0 |

### <Preparation Method>

The ingredients (2) to (4) were mixed with a stirrer followed by gradually adding the ingredient (5) and mixing them until homogeneous. To the mixture was added the ingredients (6), (7) and (1) which had been previously mixed, and they were kneaded to obtain dough. The dough was allowed to stand in a refrigerator for 30 minutes, shaped into an appropriate form, and baked in an oven to obtain cookies.

### Example 3: Gummi Candies

### <Formula>

| Ingredient | | Blending amount |
|---|---|---|
| (1) | Hot water extract of Astilbe thunbergii | 2.5 |
| | (prepared in Test Example 1) | |
| (2) | 5-Times concentrated apple juice | 45.0 |
| (3) | Honey | 41.5 |
| (4) | Lemon squeeze | 5.0 |
| (5) | Gelatin | 6.0 |
| (6) | Cinnamon | q.s. |

| | |
|---|---|
| Total | 100.0 |

### <Preparation Method>

The ingredients (1) to (4) were heated and mixed, and the ingredients (5) and (6) were added thereto and heated and mixed until homogenous. The mixed liquid was poured into molds and cooled at 4° C. for 1 hour. Thereafter, the contents were removed from the molds to obtain gummy candies.

### Example 4: Tablet-Type Supplements

### <Formula>

| Ingredient | | Blending amount |
|---|---|---|
| (1) | Hydrous ethanol extract of Astilbe | 10.0 |
| | thunbergii (prepared in Test Example 2) | |
| (2) | Microcrystalline cellulose | 75.0 |
| (5) | Sodium ascorbate | 10.0 |
| (6) | Glycerin fatty acid ester | 3.0 |
| (7) | Talc | 1.8 |
| (8) | Sodium stearate | 0.2 |

| | |
|---|---|
| Total | 100.0 |

### <Preparation Method>

The ingredients (1) to (8) were homogeneously mixed and tableted using a single punch tableting machine to obtain tablets having a diameter of 5 mm and a mass of 15 mg.

### Example 5: Granule-Type Supplements

### <Formula>

| Ingredient | | Blending amount |
|---|---|---|
| (1) | Hydrous ethanol extract of Astilbe | 15.0 |
| | thunbergii (prepared in Test Example 2) | |
| (2) | Ascorbic acid | 25.0 |
| (3) | d-α-Tocopheryl acetate | 1.5 |
| (4) | Powdered reduced maltose starch syrup | 54.0 |
| (5) | Aspartame | 0.6 |
| (6) | Hydroxypropyl cellulose | 1.5 |
| (7) | Riboflavin butyrate | 0.2 |
| (8) | Sucralose | 0.2 |
| (9) | Sucrose fatty acid ester | 2.0 |

| | |
|---|---|
| Total | 100.0 |

### <Preparation Method>

A mixture obtained by mixing the ingredients (1) to (6) was mixed with a solution of the ingredients (7) and (8) dissolved in 25 ml of ethanol, kneaded and then granulated using an extrusion granulator. The ingredient (9) was added to and mixed with the resulting granulated materials (granules) to granule-type supplements.

1. A polysaccharide digestion inhibitor comprising an Astilbe thunbergii extract as an active ingredient.

2. The polysaccharide digestion inhibitor according to claim 1, wherein the inhibitor is α-amylase inhibitor and/or α-glucosidase inhibitor.

3. The polysaccharide digestion inhibitor according to claim 1, wherein the extract is an extract with water and/or an organic solvent.

4. A food and drink comprising the polysaccharide digestion inhibitor according to claim 1.

5. An oral composition comprising the polysaccharide digestion inhibitor according to claim 1.

6. The polysaccharide digestion inhibitor according to claim 2, wherein the extract is an extract with water and/or an organic solvent.

7. A food and drink comprising the polysaccharide digestion inhibitor according to claim 2.

8. A food and drink comprising the polysaccharide digestion inhibitor according to claim 3.

9. A food and drink comprising the polysaccharide digestion inhibitor according to claim 6.

10. An oral composition comprising the polysaccharide digestion inhibitor according to claim 2.

11. An oral composition comprising the polysaccharide digestion inhibitor according to claim 3.

12. An oral composition comprising the polysaccharide digestion inhibitor according to claim 6.
